(19) United States
(12) Patent Application Publication
(10) Pub. No.: US 2008/0097564 A1
(43) Pub. Date: Apr. 24, 2008

(54) Electrotherapeutic Device

(76) Inventor: Peter Lathrop, San Diego, CA (US)

Correspondence Address: CATALYST LAW GROUP, APC, 9710 SCRANTON ROAD, SUITE S-170, SAN DIEGO, CA, 92121, US

(21) Appl. No.: 11/572,417

(22) PCT Filed: Jul. 16, 2004
(86) PCT No.: PCT/US04/23045

§ 371 Date: Jan. 19, 2007

Related U.S. Application Data
(60) Provisional application No. 60/488,673, filed on Jul. 18, 2003.

(51) Int. Cl. A61N 1/00; A61F 5/00

(52) U.S. Cl. 607/115; 602/21; 607/145

(57) ABSTRACT

This invention relates to an electrotherapeutic device useful for treating a variety of aspects associated with Carpal Tunnel Syndrome. The device is a TENS-like unit that is miniaturized, comfortable and unobtrusive, thereby allowing for unencumbered performance of daily activities. The device houses an electronic circuit comprising optimally placed electrodes and a microprocessor preprogrammed to deliver an optimal stimulus pulse protocol whereby the stimulus pulse parameters are varied so as to deliver a series of stimulus pulses for treating all aspects of CTS, including but not limited to pain blockage, nerve regeneration, reduction in inflammation and biochemical release.

### RELATED APPLICATION

Benefit of priority under 35 U.S.C. 119(e) is claimed herein to U.S. Provisional Application No. 60/488,673, filed Jul. 18, 2003. The disclosure of the above referenced application is incorporated by reference in its entirety herein.

### BACKGROUND OF THE INVENTION

Carpal Tunnel Syndrome (CTS) is the trapping of the median nerve in the wrist. Nerves are soft structures and they can become trapped at various sites in the body. The median nerve is one of the many controlling the muscles of the arm and hand. It also relays sensation from the skin on the back of the thumb, index and middle finger, and also from half of the ring finger. It runs from the elbow through the forearm to enter the wrist and hand on the same side as the palm.

Although the nerve can be damaged anywhere along its course, it is most commonly compressed at the point it enters the wrist. Here the nerve lies in a tunnel (hence the use of the term Carpal Tunnel), the floor of which is made up of bones and tendons in the wrist, and the ceiling is a band of tendon. If this “tunnel”—the carpal tunnel—becomes compressed, the median nerve is trapped and symptoms can occur.

Commonly, people with Carpal Tunnel Syndrome develop painful sensations described as “pins and needles” in the wrist and hand. The painful sensations can be most severe in the thumb, index finger, and middle fingers; however, it may occur in other parts of the fingers, hand and wrist. The pain, which is usually worse at night, may also extend into the arm. These symptoms may progress to numbness in the same areas and to weakness of the hand muscles. Weakness in several of the thumb muscles makes it difficult to grasp objects between the thumb and forefingers. If the problem is not treated and the process left unchecked, these muscles may shrink through disuse.

Many patients with CTS are unable to differentiate hot from cold by touch, and experience an apparent loss of strength in their fingers. They appear clumsy in that they have trouble performing simple tasks such as tying their shoes or picking up small objects.

Swelling of the tendons that line the carpal tunnel causes CTS. Although there are many reasons for developing this swelling of the tendon, it often results from repetitive and forceful movements of the wrist during work and leisure activities.

Research conducted by the National Institute for Occupational Safety and Health (NIOSH) indicates that job tasks involving highly repetitive manual acts, or necessitating wrist bending or other stressful wrist postures, are connected with incidents of CTS or related problems. The use of vibrating tools may also contribute to CTS.

One firm estimates that it costs a company $37,000 in lost work time, medical treatments and rehabilitation for each worker who develops CTS. Workman's Compensation figures estimate $6,000 to $10,000 per case, depending on whether one or both hands are involved; and estimate the average cost of a well-managed case would be $8,000. Because the incidence of CTS continues to increase (especially in work requiring repetitive hand movements, particularly computer keyboard users), it is financially important to consider painless, non-invasive, non-surgical treatments, which are easily self administered by the patient while at home, at work or elsewhere, without sacrificing the quality of treatment.

Treatment of CTS may involve surgery to release the compression on the median nerve and/or use of anti-inflammatory drugs and hand splinting to reduce tendon swelling in the carpal tunnel. In addition to the above mentioned costs of surgery, such medical interventions have met with mixed success, especially when an affected person must return to the same working conditions. Current non-surgical treatments include: over-the-counter analgesics; steroid cortisone injections; physical therapy; chiropractic therapy; osteopathy; acupuncture; massage; homeopathy; and support braces. However, many of these non-surgical approaches do nothing to address the source of the problem.

Transcutaneous Electrical Nerve Stimulation (TENS) is an accepted and well-characterized mode of electrotherapy (Kahn, J., Principles and Practice of Electrotherapy, New York, Churchill Livingstone, 1987; Greene, R. W. et al., Transcutaneous Pain Control and/or Muscle Stimulating Apparatus, U.S. Pat. No. 4,147,171). TENS is primarily intended for pain relief via a nerve signal blocking mechanism, but it has also been used to promote healing via reduction of carpal tunnel inflammation and the appropriate release of biochemicals (“Therapeutic Goals”).

Current TENS units are designed to deliver a single current, and require manual adjustment to vary any of the parameters; (i.e., pulse amplitude, pulse width, wave form, modulation, frequency, current and pulse times). Many patients benefit from a combined therapy in which a variety of stimulus pulse parameters are used during different stages of therapy. The availability and use of combination electrotherapy methods is often critical to successfully treating CTS. Existing units require manual adjustments by the therapist or other user to achieve this combination electrotherapy approach to CTS management. Independent use of these units by a patient (per clinician instructions) as part of a complete treatment plan of managed self care, if at all feasible, is very difficult because adjusting the pulse parameters for each treatment session creates inherent variability in the magnitude and duration of said parameters. Such a problem is further exaggerated by the lay-patient who is charged with self-administering the combination electrotherapy via a personal unit. As such, the current units do not promote the current trend in the health care field toward managed self care and effective treatment. Furthermore, involvement of the lay-user is dramatically increasing as more portable TENS units enter the market. Portable TENS devices available on the market include: TENZCARE, 3M Co., St. Paul, Minn.; Premier TENS, American Imex, Irvine, Calif.; and ProTENS, NTRON, Sugarland, Tex.

In addition to the problems associated with user involvement in setting the TENS pulse parameters, there are several problems also associated with the electrodes that are used in TENS therapy. Electrodes generally require adherence to the skin using tape or another adhesive-conducting material. The tape or material becomes loose over time rendering the electrodes and therapy ineffective. This is especially true in active patients in which the activity (e.g., passive range of motion, light exercise, normal daily activities) is prescribed as part of the overall rehabilitation therapy plan. Skin irritation may also occur with the use of these electrodes as a result of reactions to the adhesive materials used.

Interferential therapy is a very effective TENS-based treatment that is highly dependant on proper electrode placement. (T. W. Wing, Interferential Therapy: How it Works and What's New, The Digests of Chiropractic Economics, May/June 1992). Electrode placement is critical to effective treatment for all TENS therapies, but due to the interplay of the electrodes in interferential therapy, placement becomes a more critical component of delivering an effective therapy. Unfortunately, patients often lack the anatomic knowledge needed to effectively place the electrodes themselves. As a result, interferential therapy is not favorable as a self treatment therapy because patients will either misalign the electrodes thereby delivering a less than optimal therapy or the patient will have to make frequent clinic visits for electrode placement, which is impractical for a variety of reasons.

Accordingly, there exists a need for a miniaturized, portable electrotherapy device capable of delivering multiple modes of stimulus pulse to a user's wrist for the purpose of treatment and therapy of CTS. The device must be unobtrusive; it must promote proper electrode placement; it must provide optimal therapy regimens through precisely varied stimulus pulse protocols; it must be comfortable enough to be worn on the body during everyday activities; and it must permit complete freedom of movement without fear that its parts will become loose or detached. The present invention fulfills these needs and provides further related advantages.

### BRIEF SUMMARY OF THE INVENTION

The present invention resides in an improved electrotherapy device which is miniaturized and unobtrusive, thereby allowing for unencumbered performance of daily activities. The device comprises a housing and an electronics circuit. In a preferred embodiment, the housing of the current device is a sleeve, preferably formed of a flexible elastic material such as neoprene material; however, a variety of other materials can be used, including, but not limited to, elastic bandage material, which are often cotton or cotton plus another substance such as polymide. The sleeve is worn around the affected wrist of a patient offering, in addition to the electrotherapy as discussed below, compression to the affected arm.

The electronic circuit comprises embedded electrodes. The electrodes are optimally placed to contact the desired anatomical area of a user suffering from Carpal Tunnel Syndrome (CTS).

In one embodiment, electrodes are connected to the electronics circuit by a pair of lead wires originating from at least one socket on the electronics circuit. In another embodiment, electrodes are in wireless communication with the electronics circuit, thereby receiving instructions wirelessly. In this embodiment, the electrode comprises a wireless transceiver and a power source. The electrodes are positioned in the sleeve in a configuration for physically contacting specific musculo-tendonous structures.

The electronic circuit of the invention also comprises a microprocessor pre-programmed to deliver an optimal stimulus pulse and a power source, preferably comprising at least one battery to provide the operational power supply for the invention.

In the embodiment wherein the electrodes communicate wirelessly with the microprocessor, both the wireless electrodes and the microprocessor further comprise a wireless transceiver; and the electrodes still further comprise a power source.

The electronics circuit comprises a microprocessor that is pre-programmed to deliver a stimulus pulse, and during the course of an electrotherapy treatment, the pre-programmed microprocessor will vary the parameters of the stimulus pulse. The stimulus pulse is based on transcutaneous electrical nerve stimulation (TENS) and includes the following parameters: pulse amplitude, pulse width, wave form, modulation, frequency and pulse time. The microprocessor will change these parameters at a precise time, to a precise degree and a precise setting thereby specifically and optimally treating a variety of the complications associated with carpal tunnel syndrome (e.g., pain, inflammation, biochemical dysregulation, and neural impulse). Optimal changes in stimulus pulse parameters for treating a particular disorder are well known in the art. Changing these parameters using a microprocessor will avoid the inherent errors in timing, degree and settings associated with manual adjustment. In the preferred embodiment, these changes in the parameters of the stimulus pulse are consistent with defined stimulus pulse parameter changes as determined in the art of electrotherapy; however, any of a variety of changes is possible. The electronics circuit is embedded into the housing to prevent loosening during movement. Each pre-programmed set of parameters is directed to treat a specific physical condition associated with CTS.

In the preferred embodiment, the electrode pairs are precisely placed around the wrist so as to deliver a three dimensional quadripolar interferential microcurrent to an affected area. In addition, a single electrode is placed at or near the palm of the hand in order to facilitate the regeneration and healthy functioning of the medial nerve.

In an alternative embodiment, there is also included at least one remote electrode that is in communication with the control circuit (e.g., using lead wires or using radio frequency). Said at least one remote electrode is preferably placed at or near the trapezius muscles. The at least one remote electrode preferably receives instructions from the control circuit using a wireless communication protocol. In this embodiment the at least one remote electrode comprises a microprocessor having a wireless communication means; a power source; and an electrode. In an alternative embodiment, the at least one remote electrode is connected to the control circuit using a lead wire, and thus need only comprise an electrode.

Other features and advantages of the invention will become apparent from the following more detailed description, taken in conjunction with the accompanying drawings which illustrate by way of example, the principles of the invention.

### BRIEF DESCRIPTION OF THE FIGURES

FIGS. 1a and b are perspective views of the device in an open position.

FIG. 2 is an illustrative view of the device in use on the human wrist.

FIG. 3 is an illustrative view depicting electrode to electrode electrical contact during the delivery of three dimensional quadripolar interferential current.

FIG. 4 is a perspective view of the device in an open position and highlighting the at least one remote electrode and two alternative embodiments wherein in one alternative embodiment, said at least one remote electrode is hard wired to the remainder of the embedded electronics circuit, and wherein in a second embodiment, said at least one electrode wirelessly communicates with the remainder of the embedded electronics circuit

FIG. 5 is a block schematic representing an electronic circuit useful with the device.

FIG. 6 is a schematic representing an electronic circuit useful with the device.

### DETAILED DESCRIPTION OF THE INVENTION

As shown in the drawings for purpose of illustration, the current invention is concerned primarily with an improved electrotherapy device, generally designated in the accompanying drawings by the reference number 10. The device is specifically designed to be miniaturized, self-contained, and capable of affecting a plurality of different stimulation pulse modes for treating carpal tunnel syndrome (CTS).

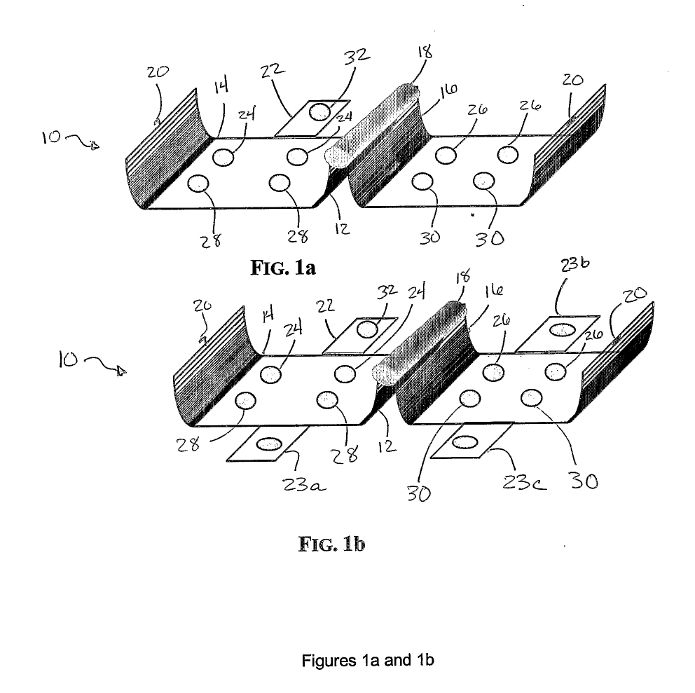

In accordance with the present invention, and as illustrated in FIGS. 1a and 1b, and applicable with respect to the preferred embodiment, the improved electrotherapy device 10 generally comprises a housing 12, which preferably further comprises a ventral-half 14 (i.e., fitting with the palmar side of the wrist) and a dorsal-half 16 (i.e., fitting with the back side of the wrist).

The housing 12 is in the form of a hinged sleeve adapted to fit around (conform anatomically to) the affected wrist and further comprises an extension to contact the palm. In the preferred embodiment, the ventral-half 14 and dorsal-half 16 of housing 12 are connected using a hinged mechanism 18. The hinged mechanism allows housing 12 to open and close around a user's wrist in a clam shell-like manner, wherein ventral-half 14 and dorsal-half 16 are capable of rotation around the longitudinal axis of said hinged mechanism 18. When in the closed position, ventral-half 14 and dorsal-half 16 are capable of fastening together using a fastener mechanism 20. Although a variety of fasteners are useful to achieve this aspect of the current invention, by way of example only, one such fastener is Shutter Pins 810 Series, (Alliance Plastics Finishing Products, Santa Fe Springs, Calif. 90670).

In an alternative embodiment, ventral-half 14 and dorsal-half 16 comprise a fastener mechanism 20 on both connecting ends. In such an embodiment, said halves are capable of complete separation from each other when open, and are directionally secured together using said fastener mechanism 20. In still a further embodiment, ventral-half 14 and dorsal half 16 comprise a retractable opening hinge on one or both connecting ends, thereby allowing for a partial separation of said halves so that a user can insert an arm into the device and then bring the two halves together by retracting and fastening said retractable opening hinge.

In an additional embodiment, the two halves, ventral-half 14 and dorsal half 16, each comprise complementary male/female attachment mechanisms. This embodiment allows for total separation of the ventral-half 14 from the dorsal-half 16 via dissociation of the male and female members. Additionally, this embodiment allows for housing inserts to be inserted in between ventral-half 14 and dorsal-half 16 thereby creating a device that accommodates a variety of sized arms. Further embodiments are readily apparent to those of skill in the art, and will be achieved all within the spirit of the current invention.

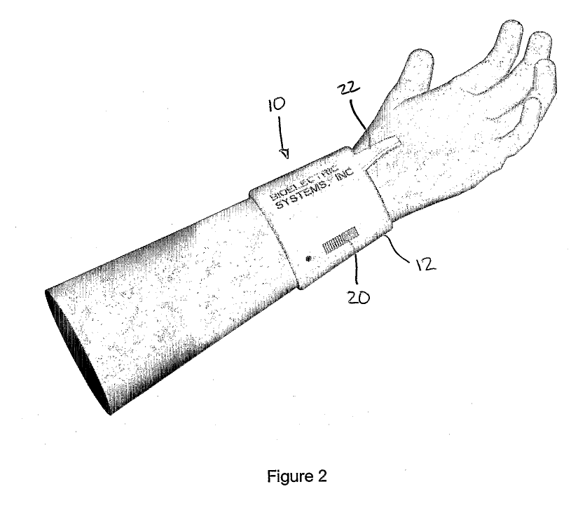

FIG. 2 illustrates one embodiment of the device in the closed position as worn on a user's wrist. In this illustration, electrotherapy device 10 is placed on a user's wrist and prong extension 22 extends into the palm of the user's hand. Other embodiments may include illustrations of additional prong extensions 23a, 23b, 23c and/or at least one remote electrode 101 (all discussed below). Although the device is shown on the wrist as applicable to treating CTS, those of ordinary skill in the art will readily adapt the device to fit a variety of areas of both human and animal bodies to treat a variety of disorders. By way of example, and not limitation, the current device can be fitted to a user's knee and the electronic circuit pre-programmed to deliver a stimulus pulse protocol useful for treating disorders of the knee.

In the preferred embodiment, ventral-half 14 of housing 12 further comprises prong extension 22, which, when housing 12 is in the closed position on a user's wrist, is in contact with the palm of said user's hand. Prong extension 22 is useful for providing splint-like support and/or may further comprise at least one electrode. Alternatively, Prongs may extend from ventral-half 14 on the forearm region and/or either end of dorsal-half 16, forming prong extensions 23a, 23b and 23c, respectively. In a further alternative embodiment, housing 12 may comprise no prong extensions.

Housing 12 is preferably constructed of a flexible and breathable material that offers the necessary support and comfort required to allow for freedom of movement yet possessing sufficient rigidity to contain the electronic components (discussed below) and to provide an additional therapeutic effect by acting as a splint to support the wrist. Such rigidity is useful in preventing the undesirable movements associated with repetitive stress injuries. In the preferred embodiment, housing 12 is constructed of neoprene; however, those of ordinary skill in the art will readily construct housing 12 from a material selected from the group consisting of, plastic, elastic, cotton, polymide, neopreme, rubber, neoprene and plaster and combinations thereof.

Housing 12 preferably comprises at least two pair of electrodes for delivery of interferential therapy and more preferably comprises at least four pair of electrodes for delivery of three dimensional quadripolar interferential therapy (generally represented in the accompanying drawings as a dark colored circle).

When housing 12 comprises two pair of electrodes, one pair of electrodes 24 is embedded in the ventral-half 14, and the other pair of electrodes 26 is embedded in the dorsal-half 16. (FIG. 1a) Electrode pairs 24 and 26 are positioned in housing 12 so as to facilitate the delivery of interferential, micro-current stimulation to the affected wrist area. In one alternative embodiment wherein housing 12 comprises four pair of electrodes, electrode pair 24 and electrode pair 28 are positioned in the ventral-half 14 and electrode pair 26 and electrode pair 30 are positioned in the dorsal-half 16 so as to facilitate the delivery of three dimensional quadripolar interferential therapy to the affected wrist area

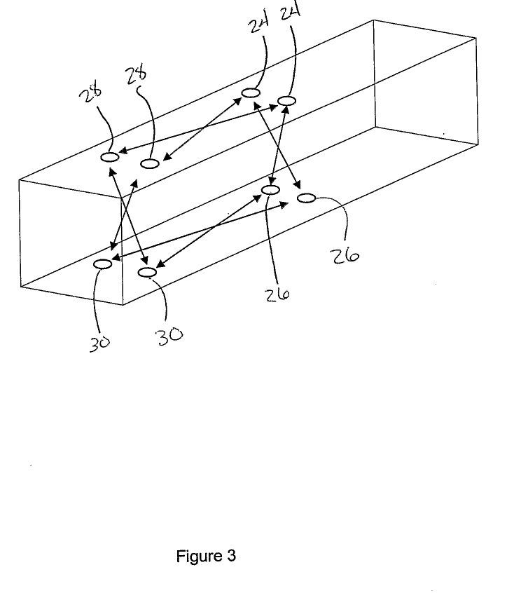

An electrode 32 can also be placed in prong extension 22 in order to deliver a stimulus pulse to the palm of a user's hand. In one embodiment, housing 12 comprises a total of nine electrodes: two pair of electrodes 24 and 28 are embedded in ventral-half 14; two-pair of electrodes 26 and 30 are embedded in dorsal half 16; and a single electrode 32 is embedded in prong extension 22. The same is true for prong extensions 23a, 23b and 23c, which may comprise electrodes. The two pair of electrodes embedded in the ventral-half 14 and the two pair of electrodes embedded in the dorsal-half 16 are positioned to facilitate the delivery of three-dimensional quadripolar, interferential, micro-current stimulation to the affected wrist. As shown in FIG. 3, quadripolar, interferential, micro-current stimulation utilizes an alternating electrical connection between electrode pairs 24-26 and 24-28, as well as a synchronized alternating current between electrode pairs 28-30 and 26-30. The principles of interferential therapies are well known in the art.

The single electrode 32 embedded in prong extension 22 is useful for stimulating the medial nerve in order to facilitate proper regeneration and function. Alternatively, any, all or a combination of prong extensions 22, 23a, 23b and/or 23c may comprise an electrode (shown as gray circles in FIG. 1b) for stimulating the medial nerve or other area of the user's forearm and hand.

Other electrode configurations are obvious to those of skill in the art in light of the current disclosure, including, but not limited to using six, eight or some other number of electrode pairs within housing 12 or using electrode pairs that span the length of housing 12 to deliver a broad width of interferential, quad interferential or three dimensional quad interferential micro-current to the affected wrist.

In the preferred embodiment, electrode pairs 24 and 28 are positioned within ventral half 14; electrode pairs, 26 and 30 are positioned within dorsal half 16; and single electrode 32 is optimally positioned within prong extension 22 to contact the wrist and palm and to deliver a desired current to the carpal tunnel and the median nerve of the wrist.

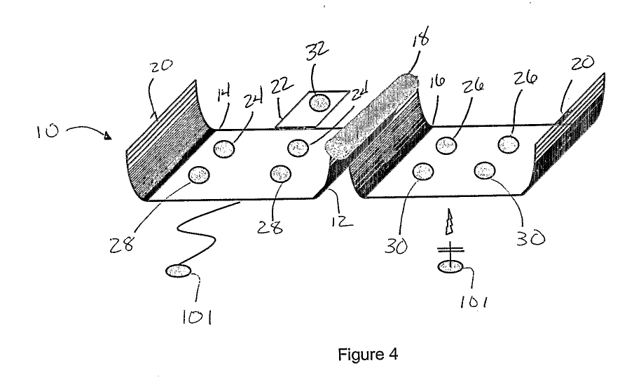

Electrotherapy device 10 may further comprise at least one remote electrode 101. FIG. 4 illustrates an at least one remote electrode 101 in one embodiment wherein a single at least one remote electrode 101 is hard wired to the electronics circuit (not shown) of ventral half 14, and illustrates an alternative embodiment wherein a single at least one remote electrode 101 is in wireless communications with the electronics circuit (not shown) of dorsal-half 16. Other embodiments are obvious to those of ordinary skill in the art.

The at least one remote electrode 101 can be placed anywhere on the body and will send a stimulating pulse to that area of the body as instructed by the electrotherapy device 10. In the preferred embodiment, at least one remote electrode 101 is placed on the trapezius muscle. More preferable, at least one remote electrode 101 is four electrodes placed on the trapezius muscle and an interferential therapy is delivered to said trapezius muscle. The trapezius muscle is preferable for placement of at least one remote electrode 101 because said muscle group has been implicated in CTS.

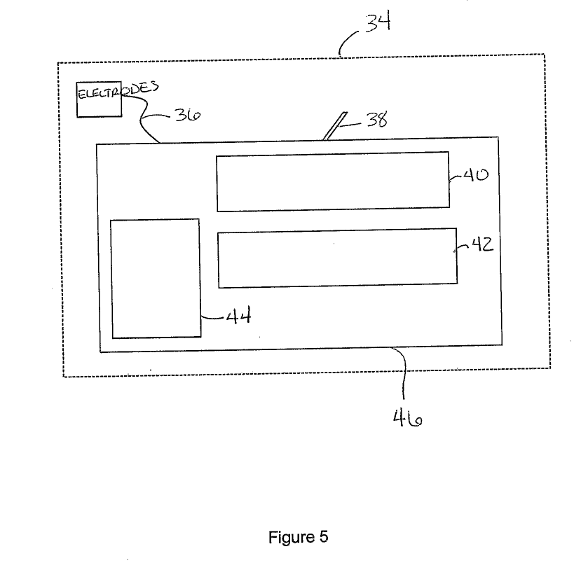

In one embodiment, as shown in FIG. 5, these electrodes (24, 26, 28, 30, 32 and 101) connect with electronics circuit 34 through lead wires 36. Both electronics circuit 34 and lead wires 36 are completely embedded in housing 12. In another more preferred embodiment the electrodes are in communication with the electronics circuit 34 using a wireless communication protocol. Wireless communication protocols are well known in the art and include without limitation those defined by the IEEE standards, for example IEEE 803.11 and IEEE 803.15, as well as others defined by protocols such as Bluetooth, Zigbee, and CDMA. In the preferred embodiment, a wireless communication protocol that is compatible with the Medical Implant Communication Service band (“MICs band”) is used because the low power requirements, falling in the range between about 260 MHz and about 700 MHz, is ideal for the medical industry.

The electronics circuit 34 is small and has low profile housing so that it is will be unobtrusive when embedded in housing 12. The electronics circuit, in its preferred embodiment shown in FIG. 5, provides at least the following: the electrodes 24, 26, 28, 30, 32 and 101 (“Electrodes” in the drawing); an on/off power switch 38, a power source 40 a microprocessor 42 and a function indicator 44.

On/off power switch 38 can be any switch known in the art that can be employed with the current invention said switch being selected from the group consisting of mechanical single pole dual throw (SPDT) switches, bio-impedance switches, push button switches and point contact switches.

The on/off power switch 38 is preferably a SPDT placed on the outer surface of housing 12 within easy access for the user. In a further embodiment, switch 38 comprises a gap in the electrical circuit that is closed by the surface of a user's skin when the housing 12 is clamped around said user's body. Point contact switches comprise a similar configuration in that the electrical circuit is open when the electrotherapy device 10 is not in use. The electric circuit is gapped across the contact point of ventral half 14 and dorsal half 16 where fastener mechanism 20 is located. When the two halves are placed in contact the electrical circuit is closed. These and other on/off switches are well known in the art and will readily be employed with the current invention.

Switch 38 controls power delivered to an electronic circuit by power source 40 contained within the electronics circuit 34. Power source 40 is preferably a primary cell battery, is more preferably a rechargeable battery, is even more preferably an embedded power source, and is most preferably a telemetric power source. In an alternative embodiment, power source 40 can be a power cord hard wired into the electronics circuit 34 and having an exposed plug for attachment into an AC wall outlet. This alternative embodiment is more useful with electrotherapy devices that will be used when a user's range of motion is limited, such as in a hospital setting.

Microprocessor 42 is preferably pre-programmed for selecting and rotating through stimulus pulse parameters. The stimulus pulse is based on transcutaneous electrical nerve stimulation (TENS) and includes the following parameters: pulse amplitude, pulse width, wave form, modulation, frequency and pulse time. The microprocessor will change these parameters at a precise time, to a precise degree and a precise setting thereby specifically and optimally treating a variety of the complications associated with carpal tunnel syndrome (e.g., pain, inflammation, biochemical dysregulation, and neural impulse). Optimal changes in stimulus pulse parameters for treating a particular disorder are well known in the art. Changing these parameters using a microprocessor will avoid the inherent errors in timing, degree and settings associated with manual adjustment.

In the preferred embodiment, microprocessor 42 is pre-programmed to deliver a current having the optimal parameters (i.e., pulse amplitude, pulse width, wave form, modulation, frequency and pulse times) to treat CTS. Using the microprocessor 42, these changes take place at precise time points during the treatment and said changes will meet the precise levels for the changed parameters. As a result, pre-programmed microprocessor 42 delivers an optimal electrotherapy treatment, which can in turn reduce recovery time, avoid symptom occurrence and reduce the cost of CTS.

In an alternative embodiment, the microprocessor can be subsequently programmed, for example by up-loading software having instructions relating to the parameters of said stimulus pulses. Those of skill in the art will readily program stimulus pulse parameters into a microprocessor.

Function indicator 44, is preferably at least one light emitting diode (LED), and is useful for indicating to the user, whether the electrotherapy device 10 is on or off, whether the power source is fully charged, and whether the electrotherapy device is conducting a current.

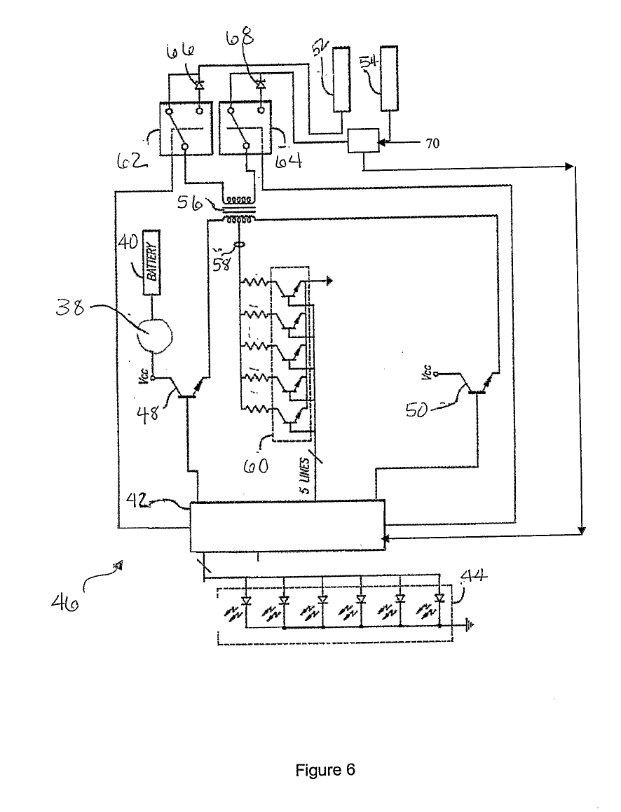

FIG. 6 shows in block diagram form the schematic for the electronics circuit 34 of the electrotherapy device 10. Power is delivered to the electronic circuit by the power source 40, preferably battery or batteries (e.g a single 9 V, or two 3 V lithium button cell batteries in series to produce 6 V). The single-pole dual-throw (SPDT) switch 38 provides an on/off function by interrupting the connection from the positive battery terminal to the electronic circuit power bus (marked as Vcc on components 48 and 50 in FIG. 6). A microprocessor 42 provides complete control over the functioning of the electronic circuit 34 by executing a series of assembly language instructions (software) stored in programmable read-only memory within the microprocessor 42. Complete digital control over operational mode selection and intensity level selection provides a greater measure of reliability, reproducibility, effectiveness and safety than what is available when the user is given control over stimulus pulse parameter selection, programming and other operating characteristics. Digital control avoids the problems of to current variability, treatment regimen inefficiency, and less than optimal overall treatment that is common, if not unavoidable, when the user is give control of the operational mode selection and intensity level selection.

The function indicator 44, preferably an LED, provides the user with information regarding whether the electrotherapy device's 10 electronics circuit 34 is on or off, is conducting current or is low on power reserves. Low power (2 mA) LEDs are preferably used for function indicator 44 to conserve battery power in power source 40. During normal operation the function indicator correspondence remains on; however, when switch 38 is in the off position, or when unit 10, for some other reason is not conducting current (e.g., dead battery), then function indicator 44 is off. In an alternative embodiment, function indicator 44 can comprise more than one LED, and various combinations of on/off for the more than one LEDs indicates a variety of messages such as type of therapy being delivered, duration or remaining time for the instant therapy, remaining power in the power supply, or stimulus pulse parameter change.

The microprocessor 42 controls stimulus pulse frequency, duration and amplitude under the direction of the associated software. The microprocessor 42 provides a logical “high” voltage to the base of either transistor 48 or transistor 50, both of which may be high current NPN or Darlington transistors, to turn the transistor on. A resistor (not shown) is placed in series at the base of each transistor to ensure that the transistor enters saturation. Transistor 48 is used to generate a positive stimulation pulse (measured at subminiature output jack 52 using subminiature jack 54 as reference). Similarly, transistor 50 is used to generate a negative stimulation pulse (measure the same as for transistor 48). Only one of these transistors is in saturation (on) at a time, and the saturated transistor serves to connect either side of a primary winding of a transformer 56 to positive voltage supply through a low resistance pathway. In an alternative embodiment, a current flow sensor 70 monitors the current from the pulse transformer 56 through the skin electrodes 52 and 54 and the user's skin. At least one implementation of this sensor is an optically coupled isolator powered by the circuitry on the other side of the transformer (not shown in FIG. 6). The sensor would provide feedback to the microprocessor block 42 relating to the resistance and other bioelectrical properties of a particular user's skin so that this block can adjust the power output of the device to obtain the desired result.

A centertap 58 of the transformer 56 is connected to the negative voltage supply (ground) through a parallel array of transistors 60, which are used to control the current through the transformer primary 56 to control the amplitude of the stimulus pulse. The transistor array 60 is contained in a single integrated circuit, and the transistors may be high current NPN (e.g., Harris semiconductor electronic part CA3081) or Darlington (e.g., Texas Instruments electronic part ULN2003). A combination of the five transistors provides predefined varied pulse amplitude (intensity) levels. Each transistor in array 60 is under separate control by the microprocessor 42, and each transistor in array 60 is operated at saturation through a series resistor (not shown) at the base of each transistor in array 60. The resistors R1 through R5 in series respectively with the collector of each array 60 transistor are selected to provide discrete steps in the peak current through the transformer 56, thus providing discrete control of the peak stimulus pulse amplitude (stimulus intensity). The values of the resistors provide linear or nonlinear discrete changes in stimulus intensity. The resistor values are chosen to provide linear changes in stimulus amplitude with resistor some resistors and to provide maximum stimulus intensity with others. A positive stimulus pulse is generated by first turning “on” transistor 48, then one of the transistors in array 60 is turned “on” to produce a particular stimulus amplitude. Both transistors remain “on” for the duration of the stimulus, which is the pulse width. The end of the pulse is generated by turning the array 60 transistor “off” then turning “off” transistor 48. A negative stimulus pulse is generated in a similar fashion using transistor 50. Through transformer action, a current pulse in the transformer primary winding produces a current pulse in the secondary winding, which is connected to the electrodes via subminiature jacks 52 and 54. The current pulse in the secondary winding is the stimulus pulse.

Solid state relays 62 and 64 selectively rectify the biphasic signal generated by transformer action using diodes 66 and 68 in order to produce the currents used in the predefined pulse stimulus modes. The solid state relays 62 and 64 are selectively activated by the microprocessor 42.

It is well known that excitable tissues will accommodate to stimulation unless the stimulation is modulated to prevent accommodation. Typical modulation schemes use amplitude modulation (the stimulation pulse amplitude is periodically changed while pulse duration and frequency are constant), pulse width modulation (pulse width is periodically changed while pulse amplitude and frequency are constant), or frequency modulation (pulse frequency is periodically changed while pulse amplitude and duration are constant). The modulation is typically represented by a triangle waveform, a sawtooth (repetitive ramps) waveform, or a sinusoidal waveform.

It is to be understood that the above description is intended to be is illustrative and not restrictive. Many embodiments will be apparent to those of ordinary skill in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

I claim:

1. An electrotherapy device for treating carpal tunnel syndrome comprising:
(a) a housing having a size and shape adapted to be worn on the body;
(b) a wrist support mechanism;
(c) at least two pair of electrodes optimally placed to deliver an electrotherapy;
(d) an electronics circuit mounted within said housing and coupled to said electrodes; and
(e) a microprocessor.

2. The electrotherapy device of claim 1, wherein the housing is plastic and is adapted to be worn around the wrist and palm of the body.

3. The electrotherapy device of claim 2 wherein the housing provides wrist support without impeding wrist function.

4. The electrotherapy device of claim 1, wherein the housing is a flexible sleeve and is adapted to be worn around the wrist and palm of the body.

5. The electrotherapy device of claim 4, wherein the wrist support mechanism further comprises a stiffening support member for providing wrist support.

6. The electrotherapy device of claim 1, wherein the first pair of electrodes is housed on the opposite side of said electrotherapeutic device from said second pair of electrodes.

7. The electrotherapy device of claim 6, wherein said first and second pairs of electrodes are optimally placed to allow for interferential currents.

8. The electrotherapy device of claim 1 wherein at least four pair of electrodes are optimally placed in said device.

9. The electrotherapy device of claim 8 wherein the four pair of electrodes are optimally placed to deliver three dimensional quadripolar interferential current.

10. The electrotherapy device of claim 1 wherein at least one electrode is optimally placed to deliver a longitudinal current to the medial nerve.

11. The electrotherapy device of claim 1, wherein at least one electrode is at least one remote electrode.

12. The electrotherapy device of claim 11, wherein the at least one remote electrode is placed on the user's shoulder muscles.

13. The electrotherapy device of claim 1, wherein the at least one electrode is physically coupled to the electronics circuit using a wire.

14. The electrotherapy device of claim 1, wherein the at least one electrode is wirelessly coupled to the electronics circuit using a wireless transceiver.

15. The electrotherapy device of claim 1, wherein the electronics circuit further comprises a power source, and wherein the power source is selected from the group consisting of primary cell batteries, rechargeable batteries, AC outlet cords with plug, embedded power sources and telemetric power.

16. The electrotherapy device of claim 1, wherein the microprocessor is pre-programmed to deliver a series of varied stimulus pulses through the electronics circuit.

17. The electrotherapy device of claim 16, wherein the microprocessor is pre-programmed to vary the parameters of a stimulus pulse, wherein the parameters of the stimulus pulse are selected from the group consisting of pulse amplitude, pulse width, wave form, modulation, frequency and pulse time.

18. The electrotherapy device of claim 16, wherein the pre-programmed variations in stimulus pulse parameters are optimal for relieving pain, for reducing inflammation, for facilitating nerve regrowth and for releasing biochemicals.

19. A method for the treatment of a disorder using electrotherapy consisting of:
(a) applying an electrotherapy device to an affected area for delivery of electrotherapy treatment;
(b) powering the electrotherapy device to deliver the treatment; and
(c) receiving an electrotherapy treatment as defined by a microprocessor wherein the parameters forming the stimulus pulses delivered are varied at precise times, precise degrees and precise settings.

20. The method of claim 19, wherein the electrotherapy device is applied to the wrist of a user.

21. The method of claim 20, wherein at least one remote electrode is applied to the shoulder muscles of a user.

22. The method of claim 19, wherein the electrotherapy treatment is an interferential therapy.

23. The method of claim 19, wherein the electrotherapy treatment is three dimensional quadripolar interferential microcurrent therapy.

24. The method of claim 19, wherein the microprocessor is pre-programmed to vary the parameters of the stimulus pulse in order to achieve an optimal electrotherapy.

25. The method of claim 24, wherein the parameters of the stimulus pulse are selected from the group consisting of pulse amplitude, pulse width, wave form, modulation, frequency and pulse time.

26. The method of claim 19, wherein the electrotherapy is for the treatment of carpal tunnel syndrome.

27. The method of claim 26, wherein the variations in stimulus pulse parameters are optimal for relieving pain, for reducing inflammation, for facilitating nerve regrowth and for releasing biochemicals.
